(19) United States
(12) Patent Application Publication
(10) Pub. No.: US 2022/0233309 A1
(43) Pub. Date: Jul. 28, 2022

(54) Transcatheter Heart Valves and Methods to Reduce Leaflet Thrombosis

(71) Applicant: Georgia Tech Research Corporation, Atlanta, GA (US)

(72) Inventors: Ajit Yoganathan, Atlanta, GA (US); Vahid Sadri, Atlanta, GA (US); Phillip Michael Trusty, Atlanta, GA (US)

(21) Appl. No.: 17/608,400

(22) PCT Filed: Apr. 7, 2020
(86) PCT No.: PCT/US20/26991

§ 371 Date: Nov. 2, 2021

Related U.S. Application Data
(60) Provisional application No. 62/851,383, filed on May 22, 2019.

(51) Int. Cl. A61F 2/24

(52) U.S. Cl. CPC A61F 2/2433; A61F 2/2418; A61F 2250/0098; A61F 2250/001; A61F 2/2436

(57) ABSTRACT

Systems and methods for replacing defective heart valves are disclosed. The systems and methods include providing a valve with a tubular frame, a plurality of valve leaflets, and an expanding member. The expanding member has a collapsed and an expanded configuration. In a collapsed configuration, the expanding member can be disposed within a catheter so that the valve can be percutaneously positioned at the defective heart valve. In the expanded configuration, the expanding member can open to exert a force on one or more native valve leaflets. By exerting the force on the native valve leaflets, the expanding member can press the native valve leaflets away from a surface of the tubular frame. Pushing the native valve leaflets from the tubular frame enables blood flow through the tubular frame and decreases the risk of thrombosis between the valve leaflets and the tubular frame.

### CROSS-REFERENCE TO RELATED APPLICATIONS

This Application claims priority, and benefit under 35 U.S.C. § 119(e), to U.S. Provisional Patent Application No. 62/851,383, filed 22 May 2019, the entire contents of which is hereby incorporated by reference as if fully set forth below.

### FIELD OF THE DISCLOSURE

Embodiments of the present disclosure relate generally to transcatheter heart valves and, more particularly, to transcatheter heart valves with expanding members to displace native heart valve leaflets.

### BACKGROUND

Aortic stenosis (AS) is the most prevalent valvular heart disease in developed countries and high mortality is associated with untreated severe AS. Patients diagnosed with moderate or severe AS undergo surgical aortic valve replacement (SAVR); approximately 67,500 surgeries are performed annually in the US. In recent years, transcatheter aortic valve replacement (TAVR) has emerged as a safe and effective alternative treatment for patients with severe, symptomatic AS and who are deemed intermediate or high surgical-risk. TAVR is a non-surgical (percutaneous) approach to aortic valve replacement which was first successfully performed in a human in 2002. TAVR procedures are performed by navigating a catheter to the native aortic valve and remotely expanding a valve inside of the native aortic annulus. In most cases, the TAVR is much less traumatic to the patient than a SAVR.

Since the inception of TAVR, the technology has advanced to support many commercial devices in the global market. Currently, however, a limited number of replacement valves available on the market. Although the number of devices available is low, the need for TAVR devices is high. Currently, approximately 180,000 patients a year can be considered potential TAVR candidates in the European Union and in Northern-America. This number might increase upwards of 270,000 if indications for TAVR expand to low-risk patients.

Despite positive outcomes at 30 days and at one year, improved imaging via four-dimensional, volume-rendered CT (4DCT) has raised concerns of subclinical leaflet thrombosis and reduced leaflet mobility in transcatheter aortic bioprostheses. It is suggested that the rate of leaflet thrombosis in transcatheter heart valves (THV) range from 4.5% to 40%. This leaflet thrombosis is caused by a “neo-sinus” forming between the frame of the THV and the THV's replacement leaflets. Because the native leaflets can rest on the frame of the THV, a “pocket” is formed where blood is stagnant which promotes thrombosis. Valve thrombosis can lead to an earlier valve failure than structural valve deterioration alone. Lifespan of a THV is particularly important as younger, lower risk patients become candidates for the procedure. Therefore, minimizing the risk factors for early valve thrombosis is key to both preventing early THV failure and to encouraging the medical community to adopt TAVR for younger patients.

Another limitation with current THV systems is the lack of mechanisms available to control the deployment height of the device. The deployment height of the THV, and the leaflets, has significant impact on the valve's function. Slight alterations in the deployment height can affect flow to the coronary arteries and/or alter valvular hemodynamics, which can in turn affect ventricular performance, valve durability/function, and aortic wall strain. What is needed, therefore, is a THV system that reduces the occurrence of leaflet thrombosis and also aids in proper alignment in the native valve.

### SUMMARY

Embodiments of the present disclosure address these concerns as well as other needs that will become apparent upon reading the description below in conjunction with the drawings. Briefly described, of the present disclosure relate generally to transcatheter heart valves and, more particularly, to transcatheter heart valves with expanding members to displace native heart valve leaflets.

An exemplary embodiment of the present invention provides a valve. The valve can include a tubular frame comprising an outer surface and defining an inner lumen, the tubular frame having a length along a longitudinal axis of the tubular frame, the length extending from a first end to a second end of the tubular frame. The valve can include a plurality of valve leaflets disposed within the inner lumen. The valve can include an expanding member extending radially outward from the tubular frame at a position along the longitudinal axis of the tubular frame. The expanding member can exert a force on one or more defective valve leaflets when the valve is deployed.

In any embodiments described herein, the expanding member can include a plurality of arms.

In any embodiments described herein, a width of each arm of the plurality of arms can be less than or equal to 1.0 mm.

In any embodiments described herein, a width of each arm of the plurality of arms can be less than or equal to 3.0 mm.

In any embodiments described herein, each arm of the plurality of arms can be cylindrical wires.

In any embodiments described herein, each arm of the plurality of arms can have a dimeter of less than or equal to 1.0 mm.

In any embodiments described herein, each arm of the plurality of arms can have a dimeter of less than or equal to 3.0 mm.

In any embodiments described herein, the expanding member can be a continuous flange.

In any embodiments described herein, the expanding member can extend from 5 mm to 10 mm from the outer surface of the tubular frame.

In any embodiments described herein, the expanding member can extend from 10 mm to 15 mm from the outer surface of the tubular frame.

In any embodiments described herein, the plurality of valve leaflets can have a first end and a second end, wherein the first end of the plurality of valve leaflets is proximate the first end of the tubular frame, and wherein the second end of the plurality of valve leaflets extends partially between the first and second end of the tubular frame.

In any embodiments described herein, the second end of the plurality of valve leaflets can be positioned approximately halfway between the first and second end of the tubular frame.

In any embodiments described herein, the expanding member can extend from the tubular frame at a position proximate the second end of the plurality of valve leaflets.

In any embodiments described herein, the expanding member can transition between a collapsed configuration and an expanded configuration.

In any embodiments described herein, when the expanding member is in the expanded configuration, the expanding member can curve toward the second end of the tubular frame.

In any embodiments described herein, the expanding member can include one or more radiopaque markers.

In any embodiments described herein, the outer surface of the tubular frame can be defined by a lattice network.

Another exemplary embodiment of the present invention provides a sleeve for a valve. The sleeve for a valve can include a tubular frame comprising an outer surface and an inner surface. The tubular frame can have a length along a longitudinal axis of the tubular frame, the length extending from a first end to a second end of the tubular frame. The sleeve for a valve can include an expanding member extending radially outward from the tubular frame at a position along the longitudinal axis of the tubular frame. The expanding member can exert a force on a defective valve leaflet when the sleeve is deployed. The inner surface can contact an exterior surface of the valve when the sleeve is deployed.

In any embodiments described herein, the expanding member can include a plurality of arms.

In any embodiments described herein, a width of each arm of the plurality of arms can be less than or equal to 1.0 mm.

In any embodiments described herein, a width of each arm of the plurality of arms can be less than or equal to 3.0 mm.

In any embodiments described herein, each arm of the plurality of arms can be cylindrical wires.

In any embodiments described herein, each arm of the plurality of arms can have a dimeter of less than or equal to 1.0 mm.

In any embodiments described herein, each arm of the plurality of arms can have a dimeter of less than or equal to 3.0 mm.

In any embodiments described herein, the expanding member can be a continuous flange.

In any embodiments described herein, the expanding member can extend from 5 mm to 10 mm from the outer surface of the tubular frame.

In any embodiments described herein, the expanding member can extend from 10 mm to 15 mm from the outer surface of the tubular frame.

In any embodiments described herein, the expanding member can transition between a collapsed configuration and an expanded configuration.

In any embodiments described herein, when the expanding member is in the expanded configuration, the expanding member can curve toward the second end of the tubular frame.

In any embodiments described herein, the expanding member can include one or more radiopaque markers.

In any embodiments described herein, the outer surface of the tubular frame can be defined by a lattice network.

In any embodiments described herein, the inner surface of the tubular frame can include an interior attachment configured to contact the exterior surface of the valve and prevent the tubular frame from moving with respect to the valve.

Another exemplary embodiment of the present invention provides a valve system. The valve system can include a stent. The stent can include a stent frame comprising an outer surface and defining an inner lumen, the stent frame having a length along a longitudinal axis of the stent frame, the length extending from a first end to a second end of the stent frame. The stent can include a plurality of valve leaflets disposed within the inner lumen. The valve system can include a tubular frame configured to contact the outer surface of the stent frame, the tubular frame comprising an expanding member extending radially outward from the tubular frame. The expanding member can exert a force on one or more defective valve leaflets when the valve system is implanted.

In any embodiments described herein, the expanding member can include a plurality of arms.

In any embodiments described herein, a width of each arm of the plurality of arms can be less than or equal to 1.0 mm.

In any embodiments described herein, a width of each arm of the plurality of arms can be less than or equal to 3.0 mm.

In any embodiments described herein, each arm of the plurality of arms can be cylindrical wires.

In any embodiments described herein, each arm of the plurality of arms can have a dimeter of less than or equal to 1.0 mm.

In any embodiments described herein, each arm of the plurality of arms can have a dimeter of less than or equal to 3.0 mm.

In any embodiments described herein, the expanding member can be a continuous flange.

In any embodiments described herein, the expanding member can extend from 5 mm to 10 mm from the tubular frame.

In any embodiments described herein, the expanding member can extend from 10 mm to 15 mm from the tubular frame.

In any embodiments described herein, the plurality of valve leaflets can have a first end and a second end, wherein the first end of the plurality of valve leaflets is proximate the first end of the stent frame, and wherein the second end of the plurality of valve leaflets extends partially between the first and second end of the stent frame.

In any embodiments described herein, the tubular frame can be positioned on the outer surface of the stent frame at a position such that the expanding member extends from the tubular frame proximate the second end of the plurality of valve leaflets.

In any embodiments described herein, the expanding member can transition between a collapsed configuration and an expanded configuration.

In any embodiments described herein, when the expanding member is in the expanded configuration and the tubular frame is in contact with the outer surface of the stent frame, the expanding member can curve toward the second end of the stent frame.

In any embodiments described herein, the expanding member can include one or more radiopaque markers.

In any embodiments described herein, the tubular frame is defined by a lattice network.

In any embodiments described herein, an inner surface of the tubular frame can include an interior attachment configured to contact the outer surface of the stent frame and prevent the tubular frame from moving with respect to the stent frame.

Another exemplary embodiment of the present invention provides a method for replacing a defective valve. The method can include delivering a valve proximate to the defective valve. The valve can include a tubular frame comprising an outer surface and defining an inner lumen, the tubular frame having a length along a longitudinal axis of the tubular frame, the length extending from a first end to a second end of the tubular frame, the second end of the tubular frame proximate the defective valve. The valve can include a plurality of valve leaflets disposed within the inner lumen. The valve can include an expanding member having a collapsed configuration and an expanded configuration. In the collapsed configuration, the expanding member can be folded towards the second end of the tubular frame. In the expanded configuration, the expanding member can extend radially outward from the tubular frame. The method can include expanding the expanding member from the collapsed configuration to the expanded configuration. The method can include advancing the second end of the tubular frame between defective leaflets of the defective valve. The expanding member can contact the defective leaflets as the tubular frame is advanced between the defective leaflets. The method can include pushing the defective leaflets against an inner wall of a vessel via the expanding member.

In any embodiments described herein, the method can include advancing the valve between the defective leaflets until the expanding member is approximately perpendicular to the tubular frame. The method can include taking a fluorographic image of the valve to confirm the expanding member is approximately perpendicular to the tubular frame.

In any embodiments described herein, the method can include taking a fluorographic image of the valve to confirm the expanding member is approximately parallel to an annular plane.

In any embodiments described herein, the method can include repositioning the valve when the expanding member is not approximately parallel to the annular plane.

In any embodiments described herein, the expanding member can include a plurality of arms.

In any embodiments described herein, a width of each arm of the plurality of arms is less than or equal to 1.0 mm.

In any embodiments described herein, a width of each arm of the plurality of arms can be less than or equal to 3.0 mm.

In any embodiments described herein, each arm of the plurality of arms can be cylindrical wires.

In any embodiments described herein, each arm of the plurality of arms can have a dimeter of less than or equal to 1.0 mm.

In any embodiments described herein, each arm of the plurality of arms can have a dimeter of less than or equal to 3.0 mm.

In any embodiments described herein, the expanding member can be a continuous flange.

In any embodiments described herein, the expanding member can extend from 5 mm to 10 mm from the outer surface of the tubular frame.

In any embodiments described herein, the expanding member can extend from 10 mm to 15 mm from the outer surface of the tubular frame.

In any embodiments described herein, the plurality of valve leaflets can have a first end and a second end, wherein the first end of the plurality of valve leaflets is proximate the first end of the tubular frame, and wherein the second end of the plurality of valve leaflets extends partially between the first and second end of the tubular frame.

In any embodiments described herein, the second end of the plurality of valve leaflets can be positioned approximately halfway between the first and second end of the tubular frame.

In any embodiments described herein, the expanding member can extend from the tubular frame at a position proximate the second end of the plurality of valve leaflets.

In any embodiments described herein, when the expanding member is in the expanded configuration, the expanding member can curve toward the second end of the tubular frame.

In any embodiments described herein, the expanding member can include one or more radiopaque markers.

In any embodiments described herein, the outer surface of the tubular frame can be defined by a lattice network.

In any embodiments described herein, the method can include partially unsheathing the valve such that the expanding member is unsheathed, thereby allowing the expanding member to expand into its expanded configuration.

In any embodiments described herein, the tubular frame can transition between a collapsed configuration and an expanded configuration. In the expanded configuration, the outer surface of the tubular frame can expand to contact a vessel wall. The method can further include fully unsheathing the valve to allow the tubular frame to expand and contact the vessel wall.

In any embodiments described herein, the valve can include an expandable balloon disposed between the plurality of valve leaflets. The tubular frame can transition between a collapsed configuration and an expanded configuration. In the expanded configuration, the outer surface of the tubular frame can expand to contact a vessel wall. The method can include unsheathing the valve, thereby allowing the expanding member to expand into its expanded configuration. The method can include expanding the expandable balloon such that the valve expands and contacts the vessel wall. The method can include removing the expandable balloon from the valve.

In any embodiments described herein, the method can include reducing a risk of thrombosis between the plurality of valve leaflets and the tubular frame.

In any embodiments described herein, the method can include increasing a flow of blood to a coronary artery.

### BRIEF DESCRIPTION OF THE FIGURES

Reference will now be made to the accompanying figures and diagrams, which are not necessarily drawn to scale, and wherein:

FIG. 1A is a cross-sectional schematic of a prior-art transcatheter heart valve in an aorta;

FIG. 1B is top view of a prior-art transcatheter heart valve;

FIG. 2A is a cross section of an aorta;

FIG. 2B is a cross-sectional schematic of a prior-art transcatheter heart valve in an aorta;

FIG. 2C is a cross-sectional schematic of an exemplary valve, according to some embodiments of the present disclosure;

FIG. 3 is a perspective view of an exemplary valve, according to some embodiments of the present disclosure;

FIG. 4 is a side view of a collapsed valve within a catheter, according to some embodiments of the present disclosure;

FIG. 5 is a side view of a collapsed valve that is partially unsheathed, according to some embodiments of the present disclosure;

FIG. 6 is a perspective view of an exemplary valve, according to some embodiments of the present disclosure;

FIG. 7 is a side view of a collapsed valve within a catheter, according to some embodiments of the present disclosure;

FIG. 8 is a side view of a collapsed valve that is partially unsheathed, according to some embodiments of the present disclosure;

FIG. 9 is a perspective view of a valve with an expanding member that is a continuous flange, according to some embodiments of the present disclosure;

FIG. 10 is a perspective view of a sleeve for a valve, according to some embodiments of the present disclosure;

FIGS. 11A and 11B are top cross-sectional views of a valve positioned within a valve annulus, according to some embodiments of the present disclosure;

FIGS. 12A-12D depict an exemplary process for inserting and deploying a valve in a valve annulus, according to some embodiments of the present disclosure; and

FIG. 13 is a flowchart of an exemplary method for repairing a defective native valve, according to some embodiments of the present disclosure.

### DETAILED DESCRIPTION

Although certain embodiments of the disclosure are explained in detail, it is to be understood that other embodiments are contemplated. Accordingly, it is not intended that the disclosure is limited in its scope to the details of construction and arrangement of components set forth in the following description or illustrated in the drawings. Other embodiments of the disclosure are capable of being practiced or carried out in various ways. Also, in describing the embodiments, specific terminology will be resorted to for the sake of clarity. It is intended that each term contemplates its broadest meaning as understood by those skilled in the art and includes all technical equivalents which operate in a similar manner to accomplish a similar purpose.

It should also be noted that, as used in the specification and the appended claims, the singular forms “a,” “an” and “the” include plural references unless the context clearly dictates otherwise. References to a composition containing “a” constituent is intended to include other constituents in addition to the one named.

Ranges may be expressed herein as from “about” or “approximately” or “substantially” one particular value and/or to “about” or “approximately” or “substantially” another particular value. When such a range is expressed, other exemplary embodiments include from the one particular value and/or to the other particular value.

Herein, the use of terms such as “having,” “has,” “including,” or “includes” are open-ended and are intended to have the same meaning as terms such as “comprising” or “comprises” and not preclude the presence of other structure, material, or acts. Similarly, though the use of terms such as “can” or “may” are intended to be open-ended and to reflect that structure, material, or acts are not necessary, the failure to use such terms is not intended to reflect that structure, material, or acts are essential. To the extent that structure, material, or acts are presently considered to be essential, they are identified as such.

It is also to be understood that the mention of one or more method steps does not preclude the presence of additional method steps or intervening method steps between those steps expressly identified. Moreover, although the term “step” may be used herein to connote different aspects of methods employed, the term should not be interpreted as implying any particular order among or between various steps herein disclosed unless and except when the order of individual steps is explicitly required.

The components described hereinafter as making up various elements of the disclosure are intended to be illustrative and not restrictive. Many suitable components that would perform the same or similar functions as the components described herein are intended to be embraced within the scope of the disclosure. Such other components not described herein can include, but are not limited to, for example, similar components that are developed after development of the presently disclosed subject matter. Additionally, the components described herein may apply to any other component within the disclosure. Merely discussing a feature or component in relation to one embodiment does not preclude the feature or component from being used or associated with another embodiment.

To facilitate an understanding of the principles and features of the disclosure, various illustrative embodiments are explained below. In particular, the presently disclosed subject matter is described in the context of transcatheter heart valves with expanding members to displace native heart valve leaflets. The present disclosure, however, is not so limited and can be applicable in other contexts. For example, the systems and methods described herein may improve other percutaneous surgical approaches. Additionally, although reference is made herein to aortic valve replacement, the systems and methods are not limited to the aortic valve. For example, the systems and methods may also be used to replace other valves, such as the mitral, pulmonic, or tricuspid valve. The devices may also be used to repair or replace implanted bioprostheses when the prosthesis fails. Accordingly, when the present disclosure is described in the context of transcatheter heart valves with expanding members to displace native heart valve leaflets, it will be understood that other embodiments can take the place of those referred to.

As stated above, both transcatheter aortic valve replacement (TAVR) and transcatheter pulmonary valve replacement (TPVR) have become viable, and popular, alternatives to surgical replacement for moderate- to low-risk patients suffering from failed valves. Taking TAVR as an example, it is expected that almost 200,000 people a year in Europe and North America may be potential candidates for percutaneous valve replacement. This is good news for these low-risk patients, as the percutaneous approach is less invasive than surgical replacement.

Current TAVR methods include inserting a guide wire into the femoral artery. The guide wire is then fed into the aorta and through the aortic valve annulus. A catheter can then be advanced over the guidewire and to the site of the valve. A transcatheter heart valve (THV) can then be inserted through the catheter and into the aortic valve annulus. Once positioned, the catheter can be removed to deploy the THV. Some THVs are self-expanding, meaning that the valve can automatically expand into the native annulus once unsheathed. Other THVs are balloon-expanding, meaning that a balloon can be disposed in the inner frame of the THV and expanded to open the THV. Certain limitations exist with both the design of current THVs and with the method of inserting current THVs.

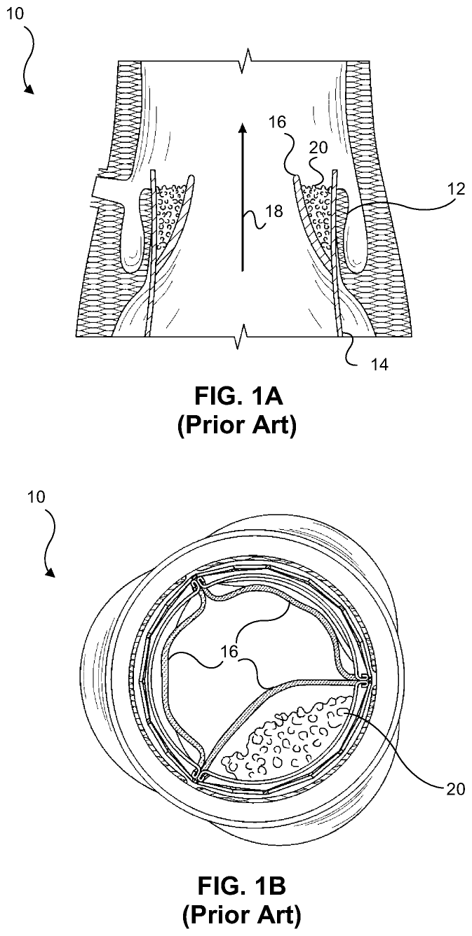

FIG. 1A is a cross-sectional schematic of a prior-art THV in an aorta 10, and the figure shows issues associated with current THV designs. As stated above, a THV is inserted into the annulus of the valve and opened between the native leaflets 12. A frame 14 of the THV abuts the native leaflets 12 and the vessel wall at the valve annulus. The frame 14 is ordinarily a lattice-type structure that enables the THV to both grip the walls of the vessel and to allow a certain amount of blood flow from inside the frame 14 to outside the frame 14. Within the frame is a plurality of THV leaflets 16 that open and close with the pumping of blood through the THV, as shown by the direction 18 of blood flow. One issue with current THV designs is the occurrence of thrombosis 20 in the area between the THV leaflets 16 and the frame 14. It has been shown that a main contributor to this formation of thromboses 20 is the lack of proper blood flow through the frame 14. As can be seen in the figure, the native leaflets 12 remain adjacent to the frame 14 and can block the flow of blood through the frame 14. As will be described in greater detail herein, this flow stagnation, or “flow stasis,” can cause the thrombus 20 to form between the THV leaflet 16 and the frame 14. FIG. 1B is top view of the existing THV shown in FIG. 1A. As can be seen the thrombus 20 can impede the opening and closing of the THV leaflets 16 and, therefore, reduce the overall motion of the THV leaflets 16.

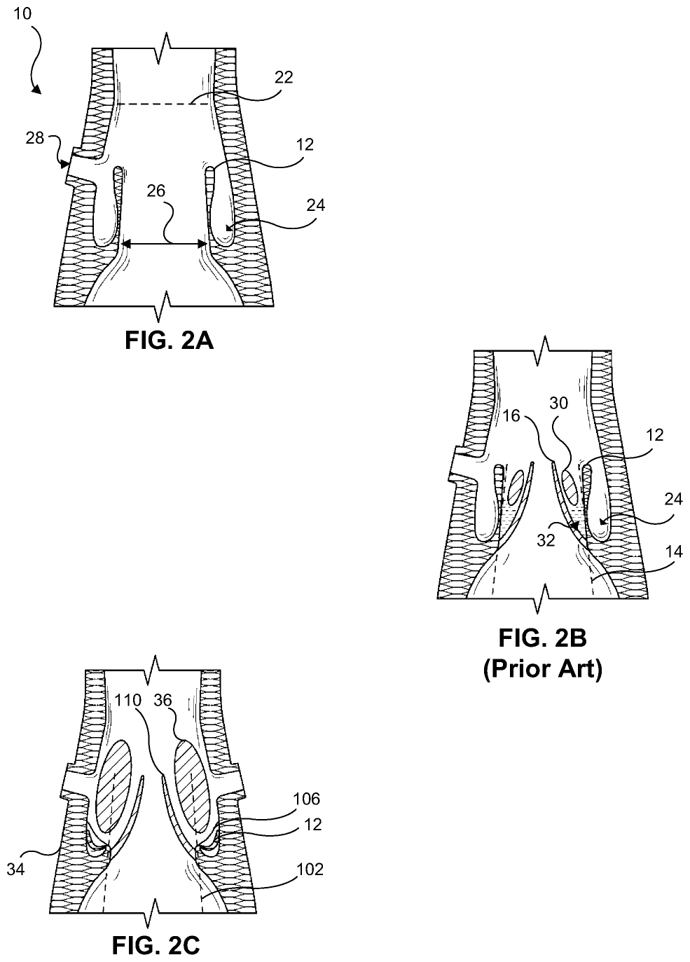

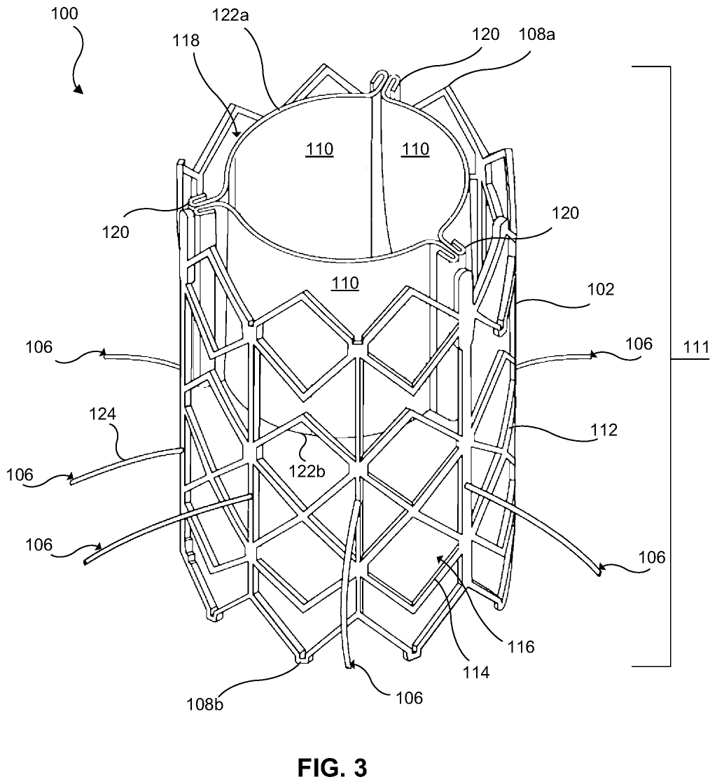

FIG. 2A is a cross section of an aorta 10, and the figure shows the various structures of the aorta 10 that are considered when implanting a THV. The sinotubular junction 22 is the transition area of the ascending aorta 10 between the tubular portion of the aorta 10 (proximal) and the sinus, for example the sinus of Valsalva 24. The valve annulus 26 is the opening between the native leaflets 12 that enables the blood to flow proximal through the valve. A THV is positioned within the valve annulus 26. Coronary arteries, for example coronary ostia 28, exit the aorta 10 at an area distal to the sinotubular junction 22. FIG. 2B is another cross-sectional schematic of prior-art THVs. When the THV is in place within the valve annulus 26, the native leaflets 12 rest on the outer surface of the frame 14. This creates a neo-sinus 30 between the native leaflet 12 and the THV leaflet 16. The neo-sinus is characterized by an area of flow stasis 32 that can lead to the thrombus described above.

FIG. 2C is a cross-sectional schematic of the valves described herein, and the figure shows a preferred design to reduce the thrombosis between the tubular frame 102 of the valve and the valve leaflets 110. As shown in the figure, if the native leaflets 12 are removed from the outer surface if the tubular frame 102 and pressed against a vessel wall 34, an area of cross flow 36 is created. This area of cross flow 36 enables blood to move freely through a porous (e.g., lattice network) tubular frame 102 such that flow stasis is not present, thereby decreasing the likelihood of thrombosis. In some examples described herein, the native leaflets 12 are pushed from the outer surface of the tubular frame 102 by an expanding member 106 extending radially outward from the tubular frame 102. In the case that the valve 100 is implanted into the aorta 10, the area of cross flow 36 can also increase blood flow to the coronary arteries. Again, it will be understood that the devices described herein can be used in valves other than the aorta, but the aorta provides a good representation of how the devices can be used within a patient.

Various devices and methods are disclosed for providing and delivering valves with expanding members to displace native valve leaflets, and exemplary embodiments of the devices and methods will now be described with reference to the accompanying figures.

FIG. 3 is a perspective view of an exemplary valve 100, according to some embodiments of the present disclosure. A valve 100 can have a tubular frame 102. The tubular frame 102 is the outer shell of the valve 100 that ultimately fits within the annulus of the native valve. It is contemplated that the tubular frame 102 can have a cylindrical shape, as shown in the figure. It is also contemplated that the tubular frame 102 can have a flare either at a first end 108a or a second end 108b of the tubular frame 102. For example, it is contemplated that the tubular frame 102 can have an hourglass shape, wherein the narrower midsection is inserted proximate the valve annulus. The first end 108a can then flare proximate the valve annulus, near the sinotubular junction; the second end 108b can flare distal to the annulus so as to fit the anatomy of the vessel. It is also contemplated that a tubular frame 102 can have only one flare, either above or below the valve annulus.

A tubular frame 102 can have a collapsed configuration and an expanded configuration. If the valve is to be used in a transcatheter approach, the valve can have a collapsed configuration that is able to be inserted through the catheter and an expanded configuration to expand and fill the valve annulus. Typical catheters for TAVR have inner diameters ranging from approximately 3.00 mm to approximately 8.00 mm. Accordingly, it is contemplated that, when the tubular frame 102 is in a collapsed configuration, the valve 100 can have an overall dimeter of from approximately 3.00 mm to approximately 8.00 mm. The native valve annulus of humans can range from approximately 15 mm to approximately 30 mm. Accordingly, it is contemplated that, when the tubular frame 102 is in an expanded configuration, the tubular frame 102 can have a diameter of from approximately 15 mm to approximately 30 mm. When considerations are made for manufacturing the device, it may be beneficial to develop a range of devices that can fit in different sized annuli. For example, a manufacturer could design a plurality of valves 100 sizes such that a physician could pick the size appropriate for the individual patient. As used herein, the terms “about” or “approximately” for any numerical values or ranges indicate a suitable dimensional tolerance that allows the part or collection of components to function for its intended purpose as described herein. More specifically, “about” or “approximately” can refer to the range of values ±20% of the recited value, e.g. “about 90%” can refer to the range of values from 71% to 99%; “approximately 15 mm” can refer to the range of value from 12 mm to 18 mm.

It is contemplated that the tubular frame 102 can be either a self-expanding construct or a balloon-expanding construct, as described above. For self-expanding constructions, the tubular frame 102 can be made from a material capable of recovering its shape automatically once unsheathed. In some examples, the material can be made from a shape memory material, such as Nitinol, and the expanded configuration for a tubular frame 102 can be made by heat setting the material to the expanded configuration. In either self-expanding or balloon-expanding constructs, the tubular frame 102 can include, but is not limited to, Nitinol, stainless steel, MP35N, tungsten, cobalt chromium, and/or the like or any combination or alloy thereof. It is also contemplated that the tubular frame 102 can include polymers, including but not limited to polyamide, polyether ether ketone, and the like.

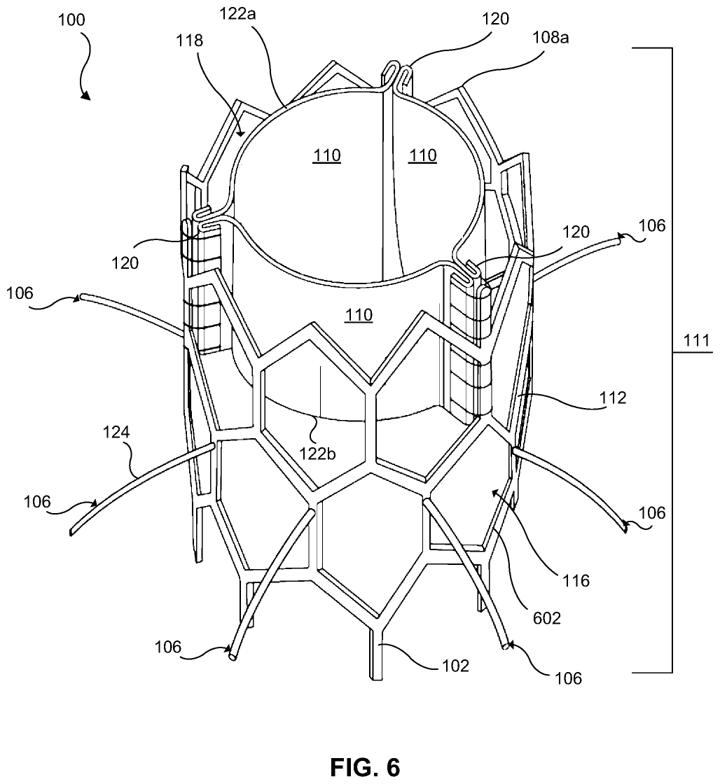

As described above, one way to decrease the occurrence of leaflet thrombosis is to promote blood flow through the tubular frame 102 or, in other words, to decrease flow stasis between the valve leaflets 110 and the tubular frame 102. Accordingly, the outer surface 112 of the tubular frame 102 can be porous or otherwise allow flow through the feature. The outer surface 112 of the tubular frame 102 can be a braided tube, laser cut metallic tube, laser cut polymeric tube and/or the like. In some examples, the outer surface 112 of the tubular frame 102 can be defined by a lattice network, as shown in FIG. 3. A lattice network can be defined by any number of shapes. FIG. 3 shows an exemplary lattice network having a plurality of diamond sections 114. The diamond sections 114 can provide pores 116 to allow blood to flow from the inner lumen 118 of the tubular frame 102 to a position outside of the tubular frame 102. The diamond sections 114 can also facilitate the expansion of the tubular frame 102 from its closed configuration to expanded configuration. Other lattice networks are also contemplated, for example a honeycomb structure (as shown in FIGS. 6-10), triangles, and/or the like.

The tubular frame 102 can define an inner lumen 118. Inside the inner lumen 118 can be a plurality of valve leaflets 110. In some examples, the valve 100 can include three valve leaflets 110, as shown in the figure, which corresponds with the anatomy of a native aortic valve. It is contemplated that a configuration could be provided wherein the valve 100 has only two valve leaflets 110 and can operate in the same manner as a three-leaflet configuration, consider for example if the valve 100 is replacing a bicuspid valve. The materials used for valve leaflets 110 can include animal materials, including but not limited to porcine pericardium tissue or allograft human tissue. The valve leaflets 110 can include synthetic polymers, engineered tissues, and/or the like. In some examples, each valve leaflet 110 can be connected to the tubular frame 102 via attachment arms 120. The attachment arms 120 can include adhesives to connect the valve leaflets 110 to the tubular frame 102, or the valve leaflets 110 can be connected mechanically, for example by sutures, hooks, wire loops, or other clasps that hold the valve leaflets 110 to the tubular frame 102.

In some examples, the valve leaflets 110 can extend the entire length 111 of the tubular frame 102. In other examples, it is contemplated that the length of the leaflets 110 is shorter than the length 111 of the tubular frame 102. For example, it is contemplated that the leaflets 110 can be positioned at some point in between the first end 108a and second end 108b of the tubular frame 102. In some examples, the valve leaflets 110 can have a first end 122a that is proximate the first end 108a of the tubular frame 102, and the valve leaflets 110 can have a second end 122b that ends at a position between the first end 108a and second end 108b of the tubular frame 102. The second end 122b of the valve leaflets 110, for example, can be approximately halfway between the first end 108a and second end 108b of the tubular frame 102. As will be described herein, the second end 122b of the valve leaflets 110 can be located at the level of an expanding member 106. FIG. 3 shows an example where there is a gap between the tubular frame 102 and the valve leaflets 110. This gap is shown to provide a detailed view of the inner lumen 118 and other features. It is contemplated that the valve leaflets 110 take up the entire inner lumen 118 such that no gap exists. This can prevent the blood from flowing around the valve leaflets 110 instead of through the valve leaflets 110.

In some examples, a valve 100 can have an expanding member 106 extending radially outward from the tubular frame 102. The expanding member 106 can be the feature of the valve 100 that pushes the native leaflets 12 away, for example into their respective coronary cusps, so that the native leaflets do not contact the outer surface 112 of the tubular frame 102. For example, FIG. 2C depicts an exemplary expanding member 106 pushing the native leaflets 12 away from the tubular frame 102. The expanding member 106 can be a variety of shapes and designs that serve to exert a force on one or more native valve leaflets when the valve 100 is deployed within the vessel. These shapes can include arms, a continuous flange (e.g., a skirt), flange with apertures, and/or similar shapes or combinations thereof, as will be described in greater detail herein.

In some examples, the expanding member 106 can comprise a plurality of arms 124 extending from the tubular frame 102, as shown in FIG. 3. Any number of arms 124 can extend from the tubular frame 102. In some examples, the arms can be cylindrical, rectangular, flat, pig-tail, or any combination thereof. For example, the arms 124 can be cylindrical wires, as shown in FIG. 3. In some examples, the arms 124 can be designed so as to not obstruct vasculature proximate the native valve being replaced. When the valve 100 is being implanted into an aorta, for example, the arms 124 can be designed to not obstruct the flow of blood to the coronary arteries. This can be achieved by providing arms 124 sufficiently spaced around the tubular frame 102 so that the coronary arteries can be avoided, and this positioning can be aided by fluoroscopy. It is also contemplated that the diameter of each individual arm 124 is not greater than the diameter of the inner lumen of a coronary artery, thereby minimizing inadvertent blocking of the arteries. For example, the mean lumen diameter of a left coronary artery can be approximately 4.5 mm, and the mean lumen diameter of a right coronary artery can be from approximately 2.5 mm and approximately 4.0 mm. Accordingly, it is contemplated that the diameter (or width if the arms are not cylindrical) of each individual arm 124 can be significantly less than the average lumen diameter of coronary arties. Accordingly, the diameter (or width) of each arm 124 can be equal to or less than 3.00 mm, for example equal to or less than 1.00 mm. The length of each arm 124 can also be customized so that vasculature is not obstructed, as will be described in greater detail herein. The diameter of the arms 124 may be larger if the arms 124 do not reach the surrounding vasculature, for example when the length of each arm 124 is customized to avoid vasculature, if necessary. To this end, it is contemplated that the arms 124 could be larger than 3.00 mm.

In some examples, and as shown in FIG. 3, the placement of the expanding member 106 on the tubular frame 102 can correspond to the location of the second end 122b of the valve leaflets 110. To illustrate, a first end 122a (the top in FIG. 3) of the valve leaflets 110 can be in-plane or substantially in-plane with a first end 108a of the tubular frame 102. A second end 122b of the valve leaflets 110 can be positioned partially down the length 111 of the tubular frame 102, for example, and not limitation, approximately half-way down the frame as shown in FIG. 3. In these examples, the expanding member 106 can be positioned such that it is attached to the tubular frame 102 at a position near the second end 122b of the valve leaflets 110. By positioning the expanding member 106 in-plane with the end of the valve leaflets 110, the expanding member 106 can act as a marker to show proper insertion depth of the valve 100 and as a marker to show if the valve 100 is tilted with respect to the annulus. The expanding member 106 can also include one or more radiopaque markers that assist the physician in placing the valve 100 at the proper depth and angle.

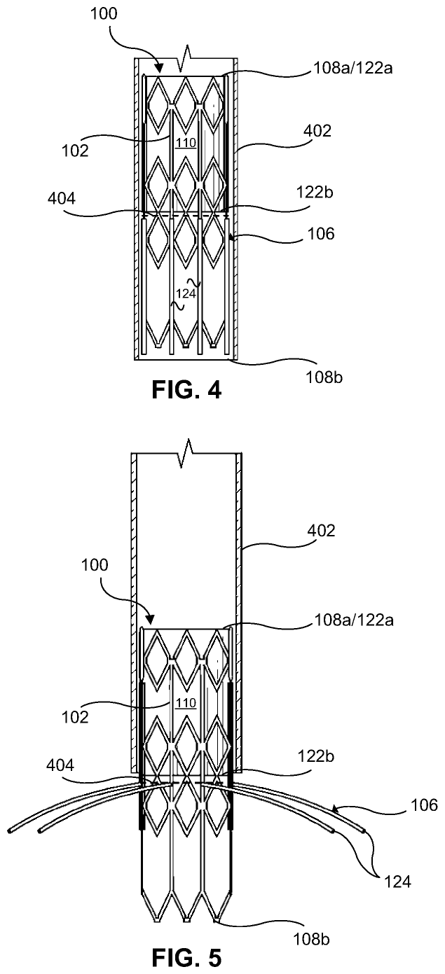

The expanding member 106 can have a collapsed configuration and an expanded configuration. FIG. 3 shows a valve 100 in an expanded configuration, which is the configuration the valve 100 takes when the valve 100 is deployed within a vessel. As described above, the valve 100 can be inserted into a vessel via a catheter. The expanding member 106 can also collapse to fit within the catheter as the valve 100 is advanced into the vessel and before the valve 100 is deployed. The collapsed configuration of an expanding member 106 is shown in greater detail in FIG. 4.

FIG. 4 is a side view of a collapsed valve 100 within a catheter 402, according to some embodiments of the present disclosure. As described above, the valve 100 can have a collapsed configuration and an expanded configuration. The collapsed configuration can be facilitated by the lattice-type network of the tubular frame 102. The expanding member 106 can also have a collapsed configuration to fit within the catheter 402. The expanding member 106 can be made from a material capable of recovering its shape automatically once unsheathed and allowed to expand. In some examples, the expanding member 106 can be made from a memory shape material, such as Nitinol, and the expanded configuration for the expanding member 106 can be made by heat setting the material to the expanded configuration prior to loading the valve 100 into the catheter 402. The expanding member 106 can also comprise, but is not limited to, stainless steel, MP35N, tungsten, cobalt chromium, and/or the like or any combination or alloy thereof. In some examples, the expanding member 106 can comprise polymers, as described above in reference to the materials for the tubular frame 102.

The example shown in FIG. 4 could also include an expandable balloon placed within the plurality of valve leaflets 110. In balloon-expandable configurations, the valve 100 can be deployed into the vessel and positioned into its implantation site. The valve 100 can then be fully unsheathed, allowing the expanding member 106 to expand into place. The tubular frame 102 can then be expanded into its expanded configuration by opening the expandable balloon, and then the balloon can be removed from inside the valve 100.

As described above, the expanding member 106 can, in some examples, enable the physician to place the valve 100 at the proper height within the annulus. To assist the proper placement of the valve 100, in some examples the expanding member 106 can be connected to the tubular frame 102 at a location near the second end 122b of the valve leaflets 110. This can provide an area 404 on the tubular frame 102 that is visible on fluoroscopy. This area 404 of where the expanding member 106 meets the tubular frame 102 can be used to check to make sure the valve is (1) inserted at the proper height with respect to the annulus and (2) not tilted with respect to the annulus.

FIG. 5 is a side view of a collapsed valve 100 that is partially unsheathed, according to some embodiments of the present disclosure. The valve 100 can be partially unsheathed to allow the expanding member 106 to open into its expanded configuration. In self-expanding constructs, the valve 100 can remain partially sheathed while the valve 100 is positioned. If the self-expanding valve 100 is partially sheathed, the tubular frame 102 can remain collapsed for proper placement. FIG. 5 also shows an expanded expanding member 106. The expanding member 106 can have a slight curve toward the second end 108b of the tubular frame 102. Although not required, a downward curve can enable the expanding member 106 to engage the native leaflets within the anatomy before the valve 100 is fully seated. The valve 100 can then be advanced farther until the expanding member 106 is approximately parallel with the annulus (or perpendicular to the tubular frame 102). This perpendicular placement of the expanding member 106, in combination with the area 404 of where the expanding member 106 meets the tubular frame 102, can be used to ensure the valve 100 is (1) inserted at the proper height with respect to the annulus and (2) not tilted with respect to the annulus. In some examples, the downward curve of the expanding member 106 can also provide mechanical feedback to the physician as the valve 100 is being inserted. The expanding member 106, for example, can provide some resistance when the expanding member 106 contacts native leaflets, and the expanding member 106 can provide even greater mechanical feedback when the expanding member 106 reaches the level of the annulus.

The expanding member 106, in some examples, can include mechanical features to prevent the valve 100 from being inserted too far into the annulus. One such mechanical feature can include providing stops, which can include tabs, at the area 404 of where the expanding member 106 meets the tubular frame 102. These stops (not shown in FIG. 5) can be placed on the tubular frame 102 at a position opposite the curvature of the expanding member 106 (i.e., on the top of the arms 124 in the figure). As the valve 100 is inserted into the anatomy, and the expanding member 106 opens and raises as the valve 100 is advanced, the stops can act to prevent the arms from raising above a certain height. The expanding member 106 can also be more rigid at its junction with the tubular frame 102 than at a point farthest from the tubular frame 102. This can be facilitated by having a thicker material proximate the tubular frame and thinner material away from the tubular frame 102. Such a configuration can enable the expanding member 106 to gently apply force to the native valve leaflets while also providing rigid support at the perimeter of the valve annulus and preventing the valve 100 from being inserted beyond the annulus.

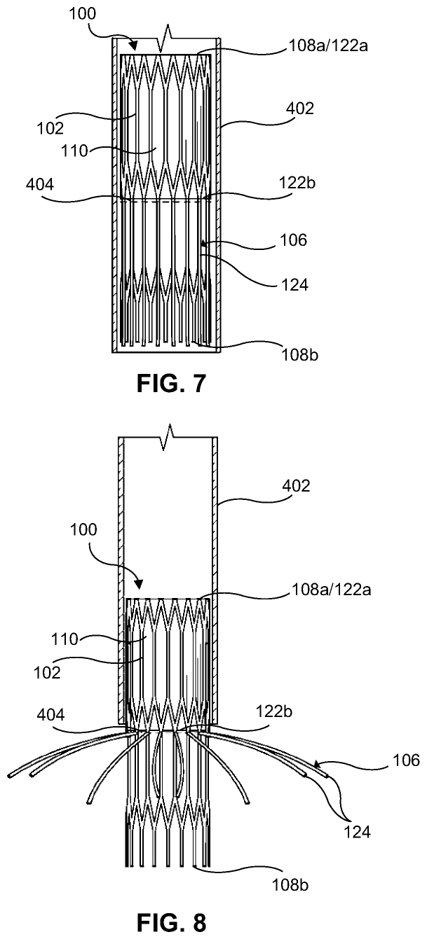

FIG. 6 is a perspective view of an exemplary valve 100, according to some embodiments of the present disclosure. As described above, the lattice network of the tubular frame 102 is not limited to the diamond lattice shown in FIGS. 3-5. It is also contemplated that the lattice network comprises a plurality of honeycomb sections 602 that define the pores 116. The honeycomb sections 602 can also facilitate the expanded configuration and the collapsed configurations described herein. FIG. 7 is a side view of a collapsed valve 100 within a catheter 402, according to some embodiments of the present disclosure. The figure shows how the honeycomb sections 602 can help the valve 100 to collapse to fit into a catheter 402. FIG. 8 is a side view of a collapsed valve 100 that is partially unsheathed, according to some embodiments of the present disclosure. FIG. 8 shows similar features to those shown in FIG. 5 but with a tubular frame 102 having a honeycomb structure.

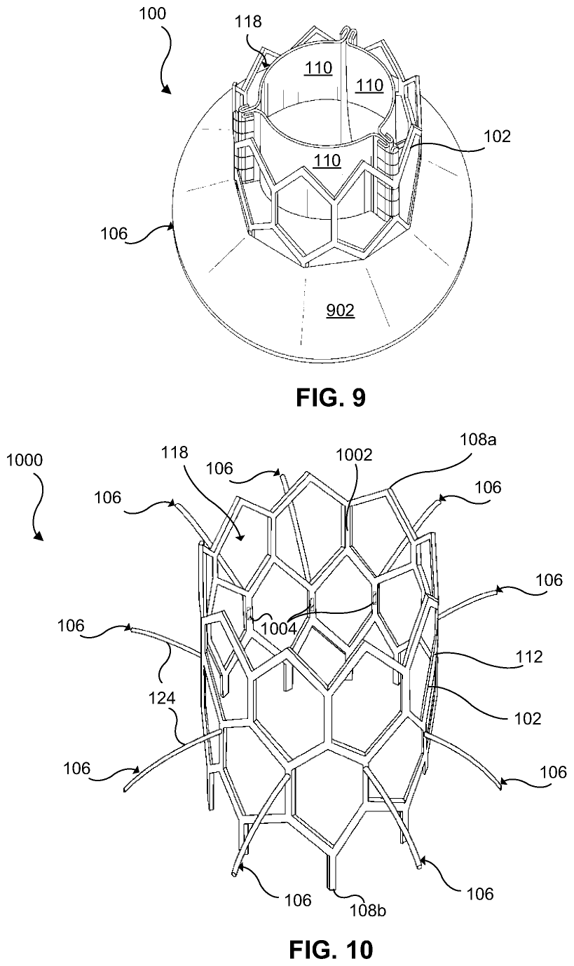

FIG. 9 is a perspective view of a valve 100 with an expanding member 106 that is a continuous flange 902, according to some embodiments of the present disclosure. As described above, the shape of the expanding member 106 can take various shapes, as more than one shape can facilitate pushing the native leaflets from the outer surface of the tubular frame 102. The previous examples, for example FIGS. 3-8, show an expanding member 106 comprising a plurality of arms 124, which is in accordance with some examples. FIG. 9 shows an expanding member 106 that is a continuous flange 902. The continuous flange 902 can be similar to a skirt around the tubular frame 102. In some examples, a continuous flange 902 can also prevent perivalvular leakage, as in the blood must pass through the valve leaflets 110 because flow around the tubular frame 102 is blocked by the continuous flange 902. It is not required that the continuous flange 902 is solid, as shown in the figure, though it can be. The continuous flange 902 can include holes, apertures, or a lattice network. The lattice network can be the same as the tubular frame 102, or the expanding member 106 could have a different lattice network than the tubular frame 102. The continuous flange 902 can also include features to provide friction against the native leaflets, for example ridges, ribs, or the like, so that the continuous flange 902 maintains a grip on the native leaflets.

FIG. 10 is a perspective view of a sleeve 1000 for a valve, according to some embodiments of the present disclosure. Certain examples of the present disclosure are able to interact with legacy THV systems to improve the legacy valve and to also decrease the risk of thrombosis in the valve. Take for example a THV that has a stent frame and a plurality of valve leaflets disposed within the stent frame. This construct does not include any feature that pushes the native leaflets away from the stent frame to encourage the area of cross flow 36 descried above in FIG. 2C. FIG. 10 shows an exemplary sleeve 1000 that can provide a solution for these legacy THVs.

A sleeve 1000 for a valve can have a tubular frame 102 and an expanding member 106. The sleeve 1000, however, can be provided without valve leaflets 110 in the inner lumen 118 of the tubular frame 102. The inner surface 1002 of the sleeve 1000 can contact the exterior surface of a stented valve when the sleeve 1000 is deployed. One way this can be performed is to first implant the sleeve 1000 into the native valve that is being replaced. The expanding member 106 of the sleeve 1000 can push the native leaflets to the vessel wall. The sleeve 1000 can then be expanded in the annulus either by self-expanding or balloon-expanding methods, as described above. A legacy THV can then be inserted into the inner lumen 118 of the tubular frame 102 and expanded to contact the inner surface 1002 of the sleeve 1000. Alternatively, the sleeve 1000 can be combined with the legacy THV prior to implanting the combined system into the patient. This can either be completed on a back table in an operating room or by manufacturing a stent with the sleeve 1000 already attached to the legacy THV.

In some examples of a sleeve 1000 for a valve, the valve can include interior attachments 1004 to contact the exterior surface of the stented valve. The interior attachments 1004 can enable the sleeve 1000 to maintain stable contact with the stented valve residing in the inner lumen 118 of the sleeve 1000. This can include preventing the sleeve 1000 from rotating with respect to the stented valve and/or preventing the sleeve 1000 from sliding axially (e.g., up and down) the length of the stented frame or vice versa. These interior attachments 1004 can include tabs, hooks, grooves to match with the lattice network of the stented valve, and/or the like.

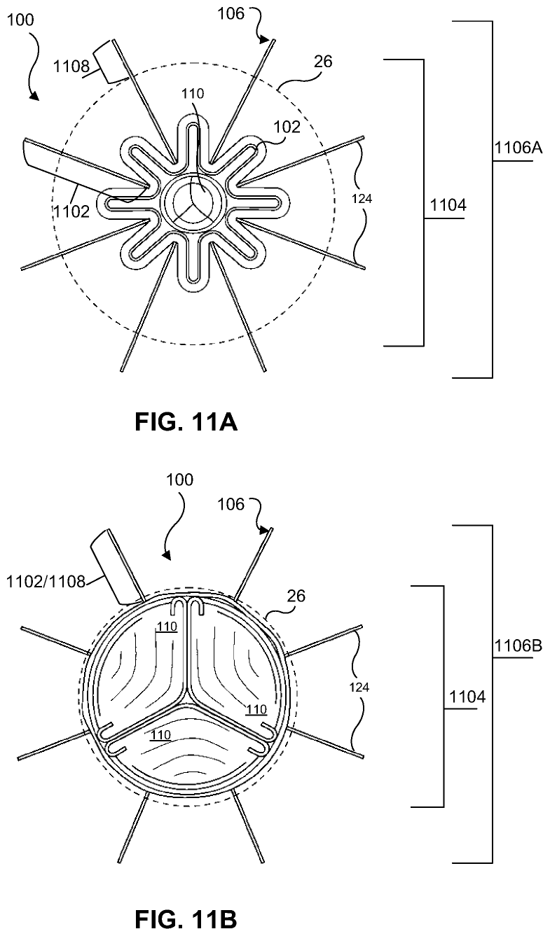

FIGS. 11A and 11B are top cross-sectional views of a valve 100 positioned within a valve annulus 26, according to some embodiments of the present disclosure. FIG. 11A depicts a tubular frame 102 in a collapsed configuration within the valve annulus 26. When the tubular frame 102 is collapsed, it can form a bunched configuration like shown in FIG. 11A. In other examples, the tubular frame 102 is not bunched, but the lattice-type network is instead collapsed upon itself, meaning the tubular frame 102 remains circular when in a collapsed configuration. Examples of this collapsed configuration are shown FIGS. 4, 5, 7, and 8. The expanding member 106 in FIG. 11A is expanded into its expanded configuration. In other words, the example shown in FIG. 11A could depict a partially-unsheathed self-expanding valve 100, where the tubular frame 102 is collapsed within a catheter and the expanding member 106 is opened to exert a force on the native valve leaflets. The example shown in FIG. 11A could also depict a fully-unsheathed balloon-expanding valve 100 that has yet to be expanded by the balloon.

The expanding member 106 can extend a length 1102 from the tubular frame 102. The exact length 1102 of the expanding member 106 depends at least on the diameter 1104 of the valve annulus 26 in which the valve 100 is being implanted. To illustrate the length 1102 of the expanding member 106, reference can be made to the diameter 1106A of the partially-expanded valve 100. The diameter 1106A in FIG. 11A refers to a length from a first end of the expanding member 106 to a second end diametrically opposite the first end. The diameter 1106A would, therefore, be the overall diameter of the valve 100 when the expanding member 106 is expanded but the tubular frame 102 remains collapsed. In any example described herein, the diameter 1106A of the collapsed valve 100 can be larger than the diameter 1104 of the valve annulus 26. This of course enables the expanding member 106 to prevent the valve 100 from passing through the valve annulus 26. At least a portion 1108 of the length 1102 of the expanding member 106 can extend beyond the valve annulus 26. To ensure the portion 1108 of the length 1102 extends beyond the valve annulus 26, it is contemplated that the expanding member 106 can extend from the tubular frame 102 at a length 1102 of from 5 mm to 15 mm (e.g., from approximately 5 mm to approximately 10 mm; or from approximately 10 mm to approximately 15 mm).

FIG. 11B depicts a tubular frame 102 and an expanding member 106 that are both in an expanded configuration within the valve annulus 26. In other words, the example shown in FIG. 11B could depict a fully-unsheathed self-expanding valve 100 or could depict a balloon-expanding valve 100 that has been expanded by the balloon. As shown in FIG. 11B, once the valve 100 is deployed and fully expanded, the tubular frame 102 is approximately the same size as the valve annulus 26, i.e., it fills the valve. Although the length 1102 of the expanding member 106 can remain the same, the portion 1108 of the length 1102 that extends beyond the valve annulus 26 can be the entire length 1102 of the expanding member 106. The expansion of the expanding member 106 can further push the native leaflets to the vessel wall. It is also contemplated that the length 1102 of the expanding member 106 can be customized so as to prevent inadvertent blocking of vasculature proximate the valve annulus 26. A shorter length 1102 can prevent inadvertent blocking of vasculature when the valve 100 is fully expanded, and a longer length 1102 can provide greater axial force to the native valve leaflets.

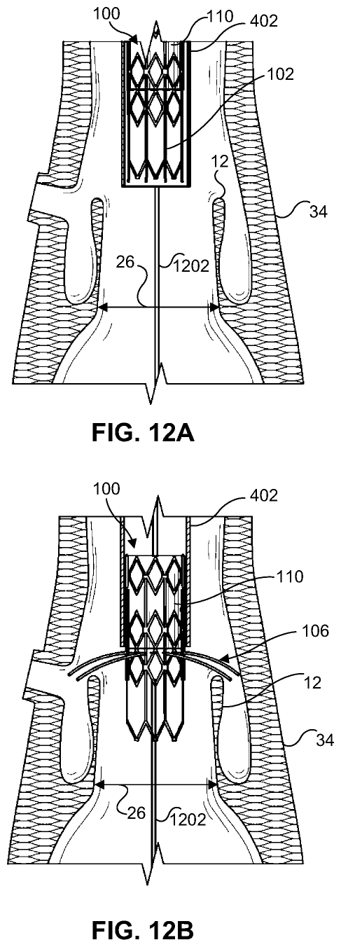

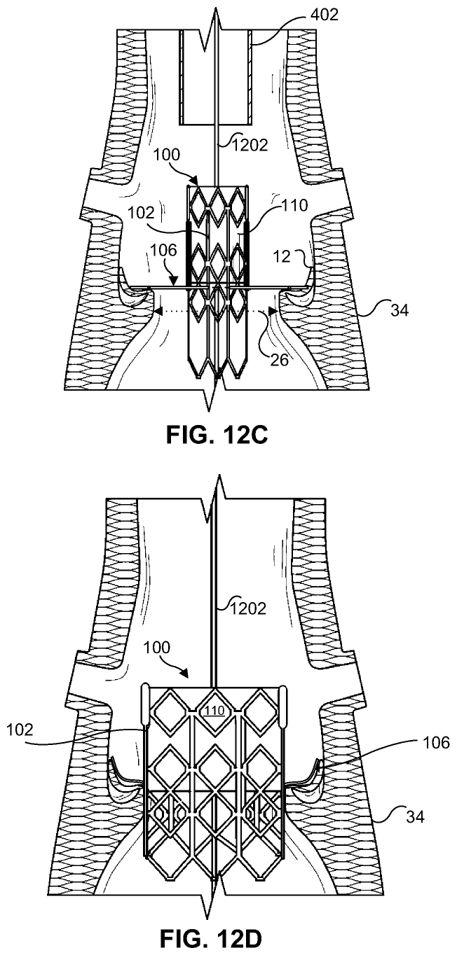

FIGS. 12A-12D depict an exemplary process for inserting and deploying a valve 100 in a valve annulus 26, according to some embodiments of the present disclosure. The figures depict a valve 100 being placed in an aortic valve. As described above, however, the present disclosure is not limited to aortic-valve replacement, and the present systems and methods can be used to replace other valves, such as the mitral, pulmonic, or tricuspid valve.

FIG. 12A depicts advancing a collapsed valve 100 via a catheter 402 proximate to a native valve that is being replaced. As can be seen in the figure, the collapsed valve 100 can be fully sheathed, and the expanding member 106 can be collapsed against the tubular frame 102. The catheter 402 and valve 100 can be inserted into the native valve by advancing the two over a guidewire 1202 placed in the native valve.

In FIG. 12B, the valve 100 is partially unsheathed to allow the expanding member 106 to expand. The example valve 100 in FIG. 12B shows a slight curve toward the bottom of the valve 100, as described above. This slight curve can enable the expanding member 106 to engage the native leaflets 12 when the valve 100 remains above the valve annulus 26. The valve 100 can next be advanced toward the valve annulus 26.

In FIG. 12C, the valve 100 is advanced into the valve annulus 26. As the expanding member 106 contacts the native leaflets 12, the expanding member 106 can begin to extend and straighten. As is shown in the figure, the expanding member 106 can displace the native leaflets 12 by pushing the native leaflets 12 against the vessel wall 34. This pushing of the native leaflets 12 can create the area of cross flow 36 (shown in FIG. 2C). By pushing the native leaflets 12 away from the tubular frame 102, the cross flow through the tubular frame 102 can decrease the chance of thrombosis in the area between the valve leaflets 110 and the tubular frame 102. As will be described in greater detail below, the present system can also be employed within an existing SAVR replacement valve. When reference is made to a native valve, it can be understood that the step can also refer to a defective, existing replacement valve.

As described herein, the position of the expanding member 106 can assist the physician in assessing proper placement of the valve 100. For example, the physician can view the placement of the valve 100 (e.g., under fluoroscopy), and when the expanding member 106 is perpendicular or is approximately perpendicular to the device (as shown in FIG. 12C), the physician can be assured the valve is placed at the proper deployment height. The physician can also confirm whether the expanding member 106 is parallel to the valve annulus 26. As described above, the expanding member 106 can be placed at a position proximate the end of the valve leaflets 110, and in this scenario, the position of the expanding member 106 can also ensure proper placement of the valve leaflets 110.

FIG. 12C also shows the valve 100 has exited the catheter 402. In this example, therefore, the tubular frame 102 can now expand automatically into its expanded configuration if the valve 100 is a self-expanding design; in balloon-expanding designs, a balloon can be expanded to expand the tubular frame 102.

In FIG. 12D, the valve 100 is deployed at the proper height in the valve annulus 26 and is fully expanded. The tubular frame 102, therefore, fills the valve annulus 26. The expanding member 106 can conform to the shape of the shape of the vessel wall 34. Once fully deployed, the physiology and fluid mechanics of the implanted valve 100 can closely mimic the physiology and fluid mechanics of the native valve.

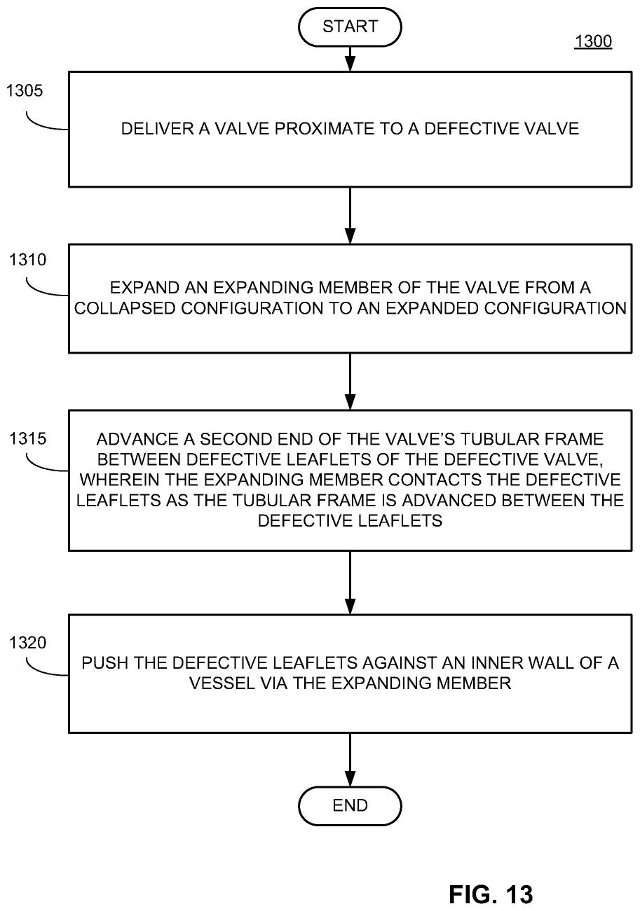

FIG. 13 is a flowchart of an exemplary method 1300 for replacing a defective valve, according to some embodiments of the present disclosure. The method 1300 can begin at block 1305, where a valve 100 is delivered proximate to the defective valve. The valve 100 delivered to the defective valve can include, for example, a tubular frame 102 comprising an outer surface 112 and defining an inner lumen 118. The tubular frame 102 can have a length 111 along a longitudinal axis of the tubular frame 102. The length 111 can extend from a first end 108a to a second end 108b of the tubular frame 102. The second end 108b of the tubular frame 102 can be proximate the defective valve. The valve 100 can also include a plurality of valve leaflets 110 disposed within the inner lumen 118. The valve 100 can also include an expanding member 106 having a collapsed configuration and an expanded configuration, wherein, in the collapsed configuration, the expanding member 106 is folded towards the second end 108b of the tubular frame 102. In the expanded configuration, the expanding member 106 can extend radially outward from the tubular frame 102.

At block 1310, the expanding member 106 is expanded from its collapsed configuration to its expanded configuration. As described herein, the expanding member 106 can be expanded independently of the tubular frame 102. This enables the expanding member 106 to open into its expanded configuration prior to fully seating the valve 100. The expanding member 106 can, therefore, exert a force on the defective valve leaflets as the valve 100 is further advanced into the defective valve (for example into the valve annulus). In some examples, the expanding member 106 can be expanded by partially unsheathing the valve 100.

At block 1315, the second end 108b of the tubular frame 102 is advanced between defective leaflets of the defective valve. The expanding member 106 can contact the defective leaflets as the tubular frame 102 is advanced.

At block 1320, the defective leaflets are pushed against a vessel wall 34 (e.g., the inner wall of the vessel) by the expanding member 106. As described herein, the defective leaflets can therefore be pushed from the outer surface 112 of the tubular frame 102, and blood can flow through the tubular frame 102. This can decrease the risk of thrombosis in the area between the valve leaflets 110 and the tubular frame 102.

The method 1300 can end after block 1320. In some examples, method 1300 can also include taking a fluorographic image of the valve 100 to confirm the expanding member 106 is approximately parallel to the annular plane of the defective valve. The fluorographic image can also confirm whether the expanding member 106 is approximately perpendicular to the tubular frame 102. This can help ensure proper valve height within the annulus. This step can also confirm the valve 100 is not tilted with respect to the annulus.

In some examples, method 1300 can also include fully unsheathing the valve 100. If the valve 100 is a self-expanding design, the full unsheathing can enable the tubular frame 102 to fully expand and contact the vessel wall. If the valve 100 is a balloon-expanding design, the balloon can be disposed between the plurality of valve leaflets 110. The entire valve 100 can be unsheathed when the valve is properly inserted, and the balloon can be expanded such that the valve 100 expands and contacts the vessel wall. The balloon can then be removed from the valve 100.

As stated above, throughout this disclosure reference has been made to delivering a valve 100 into a native valve (e.g., the native aorta). The present disclosure, however, is not so limited. It is also contemplated that the systems described herein can be implanted into an existing SAVR replacement valve. The steps described herein can be similar in this scenario, except that the expanding member 106, for example, could exert a radial force of the defective SAVR valve leaflets. Accordingly, when reference is made above to a defective valve or a defective leaflet, it can be understood to mean a defective native valve or leaflet, or a defective SAVR replacement valve or leaflet.

It is to be understood that the embodiments and claims disclosed herein are not limited in their application to the details of construction and arrangement of the components set forth in the description and illustrated in the drawings. Rather, the description and the drawings provide examples of the embodiments envisioned. The embodiments and claims disclosed herein are further capable of other embodiments and of being practiced and carried out in various ways. Also, it is to be understood that the phraseology and terminology employed herein are for the purposes of description and should not be regarded as limiting the claims.

Accordingly, those skilled in the art will appreciate that the conception upon which the application and claims are based may be readily utilized as a basis for the design of other structures, methods, and systems for carrying out the several purposes of the embodiments and claims presented in this application. It is important, therefore, that the claims be regarded as including such equivalent constructions.

Furthermore, the purpose of the foregoing Abstract is to enable the United States Patent and Trademark Office and the public generally, and especially including the practitioners in the art who are not familiar with patent and legal terms or phraseology, to determine quickly from a cursory inspection the nature and essence of the technical disclosure of the application. The Abstract is neither intended to define the claims of the application, nor is it intended to be limiting to the scope of the claims in any way. Instead, it is intended that the invention is defined by the claims appended hereto.

1. A valve comprising:
a tubular frame having a length extending from a first end to a second end of the tubular frame;
valve leaflets disposed within the tubular frame; and
an expanding member extending radially outward from the tubular frame at a position along the longitudinal axis of the tubular frame;
wherein the expanding member is configured to exert a force on one or more defective valve leaflets when the valve is deployed.

2. The valve of claim 1, wherein the tubular frame comprises an outer surface and defines an inner lumen;
wherein the tubular frame length is along a longitudinal axis of the tubular frame;
wherein the valve leaflets are disposed within the inner lumen;
wherein the valve leaflets have a first end proximate the first end of the tubular frame;
wherein the valve leaflets have a second end that extends partially between the first end and the second end of the tubular frame; and
wherein the expanding member extends from the tubular frame at a position proximate the second end of the valve leaflets.

3. The valve of claim 1, wherein the expanding member is further configured to transition between a collapsed configuration and an expanded configuration where the expanding member curves toward the second end of the tubular frame.

4. The valve of claim 1, wherein the tubular frame comprises an outer surface defined by a lattice network.

5. A valve comprising:
a tubular frame comprising an outer surface and defining an inner lumen, the tubular frame having a length along a longitudinal axis of the tubular frame, the length extending from a first end to a second end of the tubular frame;
valve leaflets disposed within the inner lumen; and
an expanding member having a collapsed configuration and an expanded configuration, wherein, in the collapsed configuration, the expanding member is folded towards the second end of the tubular frame, and wherein, in the expanded configuration, the expanding member extends radially outward from the tubular frame.

6. A valve comprising:
a tubular frame comprising an outer surface and defining an inner lumen, the tubular frame having a length extending from a first end to a second end along a longitudinal axis of the tubular frame;
valve leaflets disposed within the inner lumen; and
an expanding member extending radially outward from the tubular frame at a position along the longitudinal axis of the tubular frame;
wherein the valve leaflets have a first end proximate the first end of the tubular frame and a second end that extends partially between the first end and the second end of the tubular frame; and
wherein the expanding member:
extends from the tubular frame at a position proximate the second end of the valve leaflets; and
is configured to exert a force on one or more defective valve leaflets when the valve is deployed.

7. The valve of claim 6, wherein the expanding member comprises a plurality of arms; and
wherein each arm of the plurality of arms one or more:
has a width less than or equal to 3.0 mm;
has a width less than or equal to 1.0 mm;
has a dimeter of less than or equal to 3.0 mm;
has a dimeter of less than or equal to 1.0 mm; and
is a cylindrical wire.

8. The valve of claim 6, wherein the expanding member is a continuous flange.

9. The valve of claim 6, wherein the expanding member extends from 5 mm to 15 mm from the outer surface of the tubular frame.

10.-11. (canceled)

12. The valve of claim 6, wherein the second end of the valve leaflets is positioned approximately halfway between the first end and the second end of the tubular frame.

13. (canceled)

14. The valve of claim 6, wherein the expanding member is further configured to transition between a collapsed configuration and an expanded configuration.

15. The valve of claim 14, wherein, when the expanding member is in the expanded configuration, the expanding member curves toward the second end of the tubular frame.

16. The valve of claim 6, wherein the expanding member comprises one or more radiopaque markers.

17. The valve of claim 6, wherein the outer surface of the tubular frame is defined by a lattice network.

18. A sleeve for a valve comprising:
a tubular frame comprising an outer surface and an inner surface, the tubular frame having a length along a longitudinal axis of the tubular frame, the length extending from a first end to a second end of the tubular frame; and
an expanding member extending radially outward from the tubular frame at a position along the longitudinal axis of the tubular frame;
wherein the expanding member is configured to:
exert a force on a defective valve leaflet when the sleeve is deployed; and
transition between a collapsed configuration and an expanded configuration wherein the expanding member curves toward the second end of the tubular frame; and
wherein the inner surface is configured to contact an exterior surface of the valve when the sleeve is deployed.

19. The sleeve for a valve of claim 18, wherein the expanding member comprises a plurality of arms.

20. (canceled)

21. The sleeve for a valve of claim 19, wherein a width of each arm of the plurality of arms is less than or equal to 3.0 mm.

22. The sleeve for a valve of claim 19, wherein each arm of the plurality of arms are cylindrical wires.

23. (canceled)

24. The sleeve for a valve of claim 22, wherein each arm of the plurality of arms has a dimeter of less than or equal to 3.0 mm.

25. The sleeve for a valve of claim 18, wherein the expanding member is a continuous flange.

26. The sleeve for a valve of claim 18, wherein the expanding member extends from 5 mm to 15 mm from the outer surface of the tubular frame.

27.-29. (canceled)

30. The sleeve for a valve of claim 18, wherein the expanding member comprises one or more radiopaque markers.

31. The sleeve for a valve of claim 18, wherein the outer surface of the tubular frame is defined by a lattice network.

32. The sleeve for a valve of claim 18, wherein the inner surface of the tubular frame comprises an interior attachment configured to contact the exterior surface of the valve and prevent the tubular frame from moving with respect to the valve.

33. A valve system comprising:
a stent comprising:
a stent frame comprising an outer surface and defining an inner lumen, the stent frame having a length along a longitudinal axis of the stent frame, the length extending from a first end to a second end of the stent frame; and
a plurality of valve leaflets disposed within the inner lumen; and
a tubular frame configured to contact the outer surface of the stent frame, the tubular frame comprising an expanding member extending radially outward from the tubular frame;
wherein the expanding member is configured to exert a force on one or more defective valve leaflets when the valve system is implanted; and
wherein one or more of:
the tubular frame is positioned on the outer surface of the stent frame at a position such that the expanding member extends from the tubular frame proximate the second end of the plurality of valve leaflets;
the expanding member is configured to transition between a collapsed configuration and an expanded configuration, wherein, when the expanding member is in the expanded configuration and the tubular frame is in contact with the outer surface of the stent frame, the expanding member curves toward the second end of the stent frame; and
the tubular frame is defined by a lattice network.

34. The system of claim 33, wherein the expanding member comprises a plurality of arms.

35. (canceled)

36. The system of claim 34, wherein a width of each arm of the plurality of arms is less than or equal to 3.0 mm.

37. The system of claim 34, wherein each arm of the plurality of arms are cylindrical wires.

38. (canceled)

39. The system of claim 37, wherein each arm of the plurality of arms has a dimeter of less than or equal to 3.0 mm.

40. The system of claim 33, wherein the expanding member is a continuous flange.

41. The system of claim 33, wherein the expanding member extends from 5 mm to 15 mm from the tubular frame.

42.-46. (canceled)

47. The system of claim 33, wherein the expanding member comprises one or more radiopaque markers.

48. (canceled)

49. The system of claim 33, wherein an inner surface of the tubular frame comprises an interior attachment configured to contact the outer surface of the stent frame and prevent the tubular frame from moving with respect to the stent frame.

50. A method for replacing a defective valve comprising:
delivering the valve of claim 5 proximate to the defective valve such that the second end of the tubular frame is proximate the defective valve;
expanding the expanding member from the collapsed configuration to the expanded configuration;
advancing the second end of the tubular frame between defective leaflets of the defective valve, wherein the expanding member contacts the defective leaflets as the tubular frame is advanced between the defective leaflets; and
pushing the defective leaflets against an inner wall of a vessel via the expanding member.

51. The method of claim 50 further comprising:
advancing the valve between the defective leaflets until the expanding member is approximately perpendicular to the tubular frame; and
taking a fluorographic image of the valve to confirm the expanding member is approximately perpendicular to the tubular frame.

52. The method of claim 50 further comprising taking a fluorographic image of the valve to confirm the expanding member is approximately parallel to an annular plane.

53. The method of claim 52 further comprising repositioning the valve when the expanding member is not approximately parallel to the annular plane.

54.-59. (canceled)

60. The method of claim 50, wherein the expanding member is a continuous flange.

61. The method of claim 50, wherein the expanding member extends from 5 mm to 15 mm from the outer surface of the tubular frame.

62. (canceled)

63. The method of claim 50, wherein the valve leaflets have a first end and a second end, wherein the first end of the valve leaflets is proximate the first end of the tubular frame, and wherein the second end of the valve leaflets extends partially between the first and second end of the tubular frame.

64. The method of claim 63, wherein the second end of the valve leaflets is positioned approximately halfway between the first and second end of the tubular frame.

65. The method of claim 63, wherein the expanding member extends from the tubular frame at a position proximate the second end of the valve leaflets.

66. The method of claim 50, wherein, when the expanding member is in the expanded configuration, the expanding member curves toward the second end of the tubular frame.

67. The method of claim 50, wherein the expanding member comprises one or more radiopaque markers.

68. The method of claim 50, wherein the outer surface of the tubular frame is defined by a lattice network.

69. The method of claim 50 further comprising partially unsheathing the valve such that the expanding member is unsheathed, thereby allowing the expanding member to expand into its expanded configuration.

70. The method of claim 69, wherein:
the tubular frame is configured to transition between a collapsed configuration and an expanded configuration;
in the expanded configuration, the outer surface of the tubular frame expands to contact a vessel wall; and
the method further comprises fully unsheathing the valve to allow the tubular frame to expand and contact the vessel wall.

71. The method of claim 50, wherein:
the valve comprises an expandable balloon disposed between the valve leaflets;
the tubular frame is configured to transition between a collapsed configuration and an expanded configuration;
in the expanded configuration, the outer surface of the tubular frame expands to contact a vessel wall; and
the method further comprises:
unsheathing the valve, thereby allowing the expanding member to expand into its expanded configuration;
expanding the expandable balloon such that the valve expands and contacts the vessel wall; and
removing the expandable balloon from the valve.

72. The method of claim 50 further comprising reducing a risk of thrombosis between the valve leaflets and the tubular frame.

73. The method of claim 50 further comprising increasing a flow of blood to a coronary artery.
